(19) United States
(12) Patent Application Publication
(10) Pub. No.: US 2021/0375469 A1
(43) Pub. Date: Dec. 2, 2021

(54) APPARATUS AND METHOD FOR INDIVIDUAL HEALTH CERTIFICATION

(71) Applicant: inCode Technologies, Inc., San Francisco, CA (US)

(72) Inventors: Ricardo AMPER, San Francisco, CA (US); Jesús RUIZ, Polanco (MX); Jovan JOVANOVIC, Belgrade (RS); Sonja KESIC, Belgrade (RS); Alexey GOLUNOV, Vologda (RU); Nikola ZIVANOVIC, Belgrade (RS)

(21) Appl. No.: 17/332,759

(22) Filed: May 27, 2021

Related U.S. Application Data
(60) Provisional application No. 63/031,381, filed on May 28, 2020.

(51) Int. Cl. G16H 50/30

(52) U.S. Cl. CPC G16H 50/30; G16H 10/20

(57) ABSTRACT

A non-transitory computer readable storage medium has instructions executed by a processor to prompt a user for personal information. The user is prompted to take a self-image. The user is prompted to take an image of a user identification card. Correspondence between the self-image and a user identification card image is validated. The user record is finalized. Health test results are obtained. A digital health certificate is created if the health test results are verified. A user image from on premise location is received. Health certification criteria for the user is applied to alternately establish a criteria met state and a criteria fail state. Premise access denied messages are sent in response to the criteria fail state. A safety certification is supplied in response to the criteria met state.

### CROSS-REFERENCE TO RELATED APPLICATION

This application claims priority to U.S. Provisional Patent Application Ser. No. 63/031,381, filed May 28, 2020, the contents of which are incorporated herein by reference.

### FIELD OF THE INVENTION

This invention relates generally to enforcing health and safety measures utilizing a computer network. More particularly, this invention relates to a computer network-based system for providing a health certification for an individual.

### BACKGROUND OF THE INVENTION

Covid-19 heightened awareness of the importance of health safety at on-premise locales, such as workplaces, schools, retail establishments and the like. On-premise locales have a desire to create safe environments for users by mitigating the risk of admitting an individual that is unhealthy. To achieve this, there must be computer network-based coordination between a user device, a health certification device and an on-premise device.

### SUMMARY OF THE INVENTION

A non-transitory computer readable storage medium has instructions executed by a processor to prompt a user for personal information. The user is prompted to take a self-image. The user is prompted to take an image of a user identification card. Correspondence between the self-image and a user identification card image is validated. The user record is finalized. Health test results are obtained. A digital health certificate is created if the health test results are verified. A user image from on premise location is received. Health certification criteria for the user is applied to alternately establish a criteria met state and a criteria fail state. Premise access denied messages are sent in response to the criteria fail state. A safety certification is supplied in response to the criteria met state.

### BRIEF DESCRIPTION OF THE FIGURES

The invention is more fully appreciated in connection with the following detailed description taken in conjunction with the accompanying drawings, in which:

FIG. 1 illustrates a system configured in accordance with an embodiment of the invention.

FIG. 2 illustrates operations performed by a health certification module configured in accordance with an embodiment of the invention.

Like reference numerals refer to corresponding parts throughout the several views of the drawings.

### DETAILED DESCRIPTION OF THE INVENTION

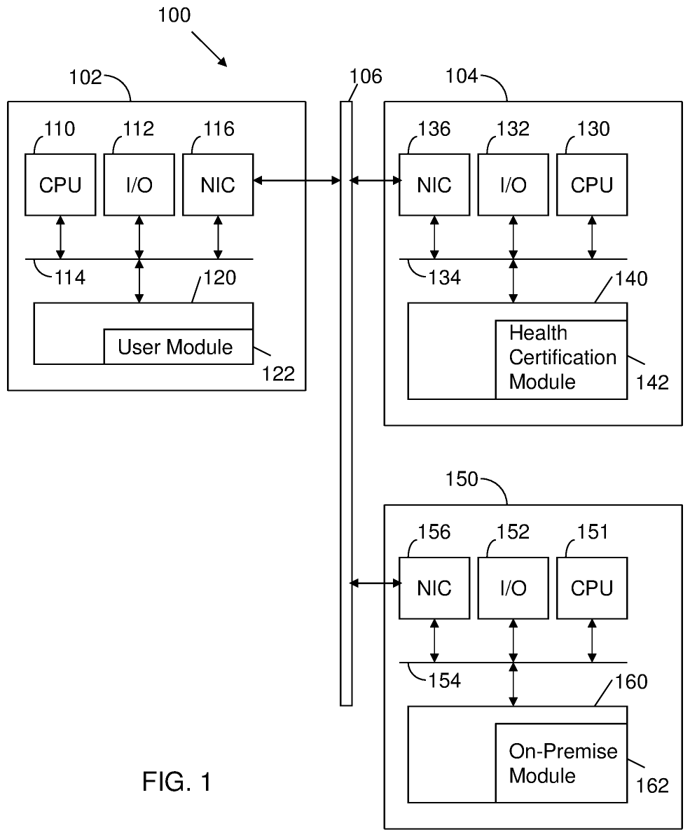

FIG. 1 illustrates a system 100 configured in accordance with an embodiment of the invention. The system 100 includes a user device 102, a health certification device 104 and an on-premise device 150 lined by a computer network 106, which may be any combination of wired and wireless networks.

The client device 102 includes a processor (e.g., central processing unit) 110, input/output devices 112 linked by a bus 114. A network interface circuit 116 is also connected to bus 114 to provide connectivity to network 106. A memory 120 is also connected to the bus 114. The memory 120 stores a user module 122 with instructions executed by processor 110 to coordinate network communications with health certification device 104 and on-premise device 150.

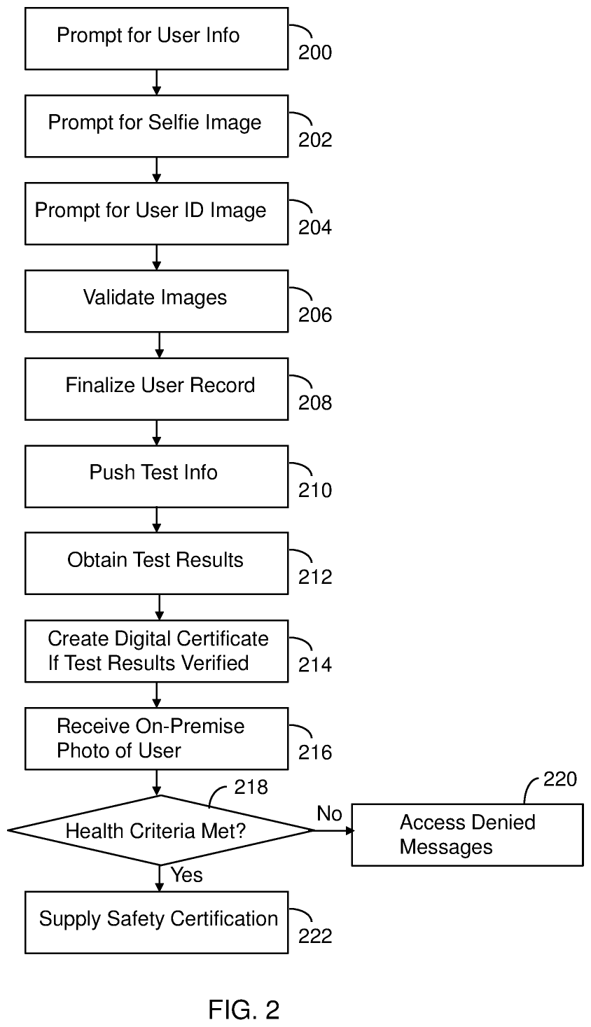

The health certification device 104 includes a processor 130, input/output devices 132, bus 134 and network interface circuit 136. A memory 140 is connected to bus 134. The memory 140 stores a health certification module 142 with instructions executed by processor 130 to implement operations disclosed in connection with FIG. 2.

The on-premise device 150 includes a processor 151, input/output devices 152, a bus 154 and a network interface circuit 156. A memory 160 is also connected to the bus 154. The memory stores instructions executed by processor 151 to implement operations disclosed herein.

FIG. 2 illustrates processing operations associated with an embodiment of the health certification module 142. Initially, a user is prompted for information 200. That is, the health certification module 142 sends prompts over network 106 to user device 102, which displays the prompts on an output device of the input/output devices 112. At a minimum, the prompts include a prompt to opt-into the system. Alternately, the prompts may solicit user name and other personal information. The user name and other personal information may alternately be secured from the on-premise module 162 which coordinates with the health certification module 142 to enroll all potential individuals that may want to enter the premise locale associated with the on-premise module 162.

The next operation of FIG. 2 is to prompt the user to take a selfie image (or self-image) 202. For example, the user may take an image of herself using a camera associated with the input/output devices 112. The user module 122 then uploads the image to the health certification module 142.

Next, the user is prompted to take an image of a user identification card 204. The user may take the image of the user identification card using a camera associated with the input/output devices 112. The user module 122 then uploads the image to the health certification module 142.

The health certification module 142 then uses facial recognition techniques to confirm that the selfie image corresponds to an image in the user identification card 206. The health certification module 142 may also scrape the user identification card for personal information associated with the user that is specified in the user identification card. Such information may be used to finalize a user record 208. The user record may also be finalized using information from an enterprise database associated with the on-premise module 162.

The health certification module 142 may then push test information 210 to the user device. The test information may specify the type of test required to enter an on-premise locale, where the test is available and similar information. The user may subsequently secure such a health test. The test results are obtained 212 by the health certification module 142. For example, the test results may be sent to user device 102, which passes them to the health certification module 142. Alternately, if appropriate permissions exist, the health certification module 142 may obtain the test results from a medical facility.

The health certification module 142 creates a digital certificate in the event that the test results are verified 214. The test results may be verified by evaluating the authenticity of the test results, the authenticity of the testing facility, and the like. The digital certificate may be sent to the user device 102 and/or on-premise device 150.

The health certification module 142 subsequently receives an on-premise photo of a user 216. For example, on-premise machine 150 may include an input device of the input/output devices 152 in the form of a kiosk with a display that provides information to a user and is capable of taking a photo of the user. The display may also include a thermal sensor to take the temperate of the user.

The health certification module 142 then determines if health criteria are met 218. If not (218—No), access denied messages 220 are sent. Such messages may be sent to the user device 102 and the on-premise device 150. If so (218—Yes), a safety certification is supplied 222. The safety certification may be sent to the user device 102 and the on-premise device 150. In sum, evaluation of the health certification criteria for the user alternately establishes a criteria met state (218—Yes) and a criteria fail state (218—No).

The health certification criteria may require positive health test results. The health certification criteria may require an on-premise image of the user with a face covering. The health certification criteria may require an on-premise sanitizer application to the user. The health certification criteria may require an on-premise safe temperature reading for the user. The health certification criteria may require a successfully evaluated user health questionnaire. The health questionnaire may be sent to the user device 102. The user answers health questions in the health questionnaire. The health question answers are then evaluated by the health certification module 142 to confirm that they meet pre-specified standards.

An embodiment of the present invention relates to a computer storage product with a computer readable storage medium having computer code thereon for performing various computer-implemented operations. The media and computer code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs, DVDs and holographic devices; magneto-optical media; and hardware devices that are specially configured to store and execute program code, such as application-specific integrated circuits (“ASICs”), programmable logic devices (“PLDs”) and ROM and RAM devices. Examples of computer code include machine code, such as produced by a compiler, and files containing higher-level code that are executed by a computer using an interpreter. For example, an embodiment of the invention may be implemented using JAVA®, C++, or other object-oriented programming language and development tools. Another embodiment of the invention may be implemented in hardwired circuitry in place of, or in combination with, machine-executable software instructions.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A non-transitory computer readable storage medium with instructions executed by a processor to:
prompt a user for personal information;
prompt the user to take a self-image;
prompt the user to take an image of a user identification card;
validate correspondence between the self-image and a user identification card image;
finalize user record;
obtain health test results;
create a digital health certificate if the health test results are verified;
receive user image from on premise location;
apply health certification criteria for the user to alternately establish a criteria met state and a criteria fail state;
send premise access denied messages in response to the criteria fail state; and
supply a safety certification in response to the criteria met state.

2. The non-transitory computer readable storage medium of claim 1 further comprising instructions executed by the processor to finalize the user record with information scraped from the user identification card.

3. The non-transitory computer readable storage medium of claim 1 further comprising instructions executed by the processor to finalize the user record with information from an enterprise database.

4. The non-transitory computer readable storage medium of claim 1 further comprising instructions executed by the processor to obtain health test results from the user.

5. The non-transitory computer readable storage medium of claim 1 further comprising instructions executed by the processor to obtain health test results from a medical facility.

6. The non-transitory computer readable storage medium of claim 1 wherein the health certification criteria include positive health test results.

7. The non-transitory computer readable storage medium of claim 1 wherein the health certification criteria include an on-premise image of the user with a face covering.

8. The non-transitory computer readable storage medium of claim 1 wherein the health certification criteria include an on-premise sanitizer application to the user.

9. The non-transitory computer readable storage medium of claim 1 wherein the health certification criteria include an on-premise safe temperature reading for the user.

10. The non-transitory computer readable storage medium of claim 1 wherein the health certification criteria include a successfully evaluated user health questionnaire.
